(19) United States
(12) Patent Application Publication
(10) Pub. No.: US 2026/0108418 A1
(43) Pub. Date: Apr. 23, 2026

(54) HEALTH CARE DEVICE AND USE THEREOF

(71) Applicant: TAIWAN OASIS TECHNOLOGY CO., LTD., New Taipei City (TW)

(72) Inventors: Ming-Shun LEE, New Taipei City (TW); Chin-Sung TSENG, New Taipei City (TW); Hsu-Hui TSENG, New Taipei City (TW); Hsien-Ching TSENG, New Taipei City (TW); Wei-Long LEE, New Taipei City (TW)

(21) Appl. No.: 19/001,070

(22) Filed: Dec. 24, 2024

(30) Foreign Application Priority Data
Oct. 21, 2024 (TW) 113140019

(51) Int. Cl. A61H 23/00; A61H 23/02

(52) U.S. Cl. CPC A61H 23/006; A61H 23/0254; A61H 2201/5005; A61H 2201/5025; A61H 2201/5053; A61H 2205/081; A61H 2205/083

(57) ABSTRACT

A health care device and an use thereof are configured to transmit a low frequency wave to a spine or navel to increase the number of endothelial progenitor cells (EPC) and to reduce the number of the circulating endothelial cells (CEC), which may reduce the risk of cardiovascular-related adverse diseases. The health care device includes a box body, a power supply, a speed controller, a rotating mechanism, a linkage mechanism, a knocker and a conical table. The power supply is electrically connected to the speed controller to output electricity to the speed controller. The speed controller is electrically connected to the rotating mechanism and controls the rotating mechanism to rotate at a predetermined speed. The rotating mechanism is connected to the knocker to drive the knocker to knock a flat surface of the conical table. The knocker generates the low frequency wave with a predetermined frequency.

### BACKGROUND OF THE INVENTION

### (a) Field of the Invention

The present disclosure relates to a health care device and a use thereof, and more particularly to the health care device and the use thereof which may increase the number of endothelial progenitor cells (EPC) and reduce the number of circulating endothelial cells (CEC) and may be a knocking device generating a low frequency wave.

### (b) Description of the Prior Art

It is known from the literature that the endothelial progenitor cells (EPC) are the progenitor cells of the vascular endothelial cells and play an important role in angiogenesis and the repair of the vascular endothelial. Therefore, increasing the number of EPC may prevent or mitigate the occurrence of the cardiovascular diseases. In contrast, the circulating endothelial cells (CEC) may be the indicator while the vascular is damaged, which may lead platelets and white blood cells to easily adhere to the wall of the blood vessels. Inflammation and fat accumulation may be generated, and the blood vessels may gradually harden. The inner diameter of blood vessels may gradually decrease. Eventually, thrombosis, arteriosclerosis and other diseases may happen. Therefore, reducing the number of CEC may reduce the risk of cardiovascular-related adverse diseases.

According to the theory of traditional Chinese medicine, the navel is the Shenque point and is the place where Ren, Du, Dai, and Chong pass through. Therefore, the whole body may be adjusted when the navel is stimulated and the internal organs are connected through meridians. Furthermore, according to the theory of Western medicine, the navel is the thinnest portion of the epidermal cuticle in abdomen and no fat is located under the navel, so the barrier function of the navel is the weakest. The skin of the navel has microcirculatory blood vessels, a vein network and the branches of the hypogastric artery. Therefore, as long as a certain effect or change is performed on the blood in the microcirculatory blood vessels of the skin of the navel, this effect or change can be introduced into the human blood circulation system. In the same way, the spine is covered by a thin epidermis, and the spine includes blood vessels and capillaries. Therefore, as long as the certain effect or change is performed on the blood in the blood vessels or capillaries in the spine, this effect or change can also be introduced into the human blood circulation system as well.

In addition, it is known from the literature that organs of the human body have inherent oscillation frequencies, such as 8-12 Hz for the head, 4-6 Hz for the chest, 5 Hz for the heart, 6-9 Hz for the abdominal cavity, 1.2 Hz for the blood flow frequency and 1.8 Hz for bone. When a portion of the human body is under the field of the low frequency wave in a range from 0 to 20 Hz (such as the bass wave or the infrasound wave (opposite to the ultrasonic wave or the ultrasonic wave higher than 20 kHz) and if the sound pressure level reaches a certain threshold, it will cause the elastic wall on the outer layer of the organs corresponding to the portion of the human body to enter a state of the vibration, and then this vibration will be transmitted to the inside of the organs. In particular, when the frequency of the bass wave is synchronized with the aforementioned oscillation frequency of the organs, they will be resonant. At this time, the organs will receive the greatest stimulation. The resonance caused by the bass wave performing on the organs will also happen on the cellular structure of the organs, such as the mitochondria of the cells. Mitochondria are responsible for intracellular oxidative phosphorylation and synthesis of adenosine triphosphate (ATP) and provide chemical energy for the activities of the cells, so Mitochondria are known as “the power station of the cells”. In addition, the resonance caused by the bass wave performing on Mitochondria may affect the binding state of some enzymes to cell membranes and also affect the activity of enzymes. At the same time, when the bass wave with a certain intensity is performed, the energy of the bass wave can be converted into thermal energy, biochemical energy and bioelectric energy, thereby directly performing on the organs. So, the damaged organs may be repaired and recovered under the stimulation of the bass wave. Using this health care device that generates the bass wave may avoid the risk of injuries caused by excessive energy generated by the high-frequency microwave oscillators.

Based on the aforementioned statement, a health care device with a low frequency wave transmitter is known currently, which is configured to transmit the low frequency wave with a specific frequency at a predetermined position away from the navel of the human body to achieve health care effects. For example, Taiwan Patent Application Publication No. TW202339822A, TW202415360A, and TW202442266A disclose a health care device which uses a low frequency wave transmitter to emit the low frequency wave with a specific frequency toward the navel of the human body. TW202339822A, TW202415360A, and TW202442266A are respectively configured to increase the number of activated T cells in the blood, the number of activated T cells and B cells in the blood, and the ability of NK cell lines to kill cancer cell lines (K562). In addition, for example, Taiwan Patent Application Publication No. TWM657715U and TWM657714U disclose that negative ions have the effects of blood purification, cell restoration, enhancement of resistance and self-healing ability, and adjustment of autonomic nerves in the human body.

However, the aforementioned health care devices that use a low frequency wave transmitter to emit the low frequency wave still need to be further improved for the intensity of the low frequency wave.

Therefore, researchers may want to develop a health care device that can emit the low frequency waves with greater intensity to increase the number of EPC and reduce the number of CEC.

### SUMMARY OF THE INVENTION

One aspect of the present disclosure is a health care device, which includes a box body and a group of components in the box body that generate the low frequency wave. The resonance cavity structure generated by the box body can cause the low frequency wave generated by the group of the components to resonate, thereby amplifying the intensity of the low frequency wave.

According to an embodiment of the present disclosure, a health care device includes a box body, a power supply, a speed controller, a rotating mechanism, a linkage mechanism, a knocker, and a conical table. The power supply, the speed controller, the rotating mechanism, the linkage mechanism, the knocker and the conical table are disposed in the box body. The power supply is electrically connected to the speed controller to output electricity to the speed controller. The speed controller is electrically connected to the rotating mechanism and controls the rotating mechanism to rotate at a predetermined speed. The predetermined speed causes the knocker to knock the conical table at a knocking frequency. The knocking frequency is in a range from 24 times/per minute to 64 times/per minute. The knocker generates a low frequency wave with a predetermined frequency. The predetermined frequency is in a range from 1.18 Hz to 1.81 Hz. The low frequency wave is a bass wave. The rotating mechanism is connected to the knocker by the linkage mechanism to drive the knocker to knock a flat surface of the conical table, such that the conical table transmits the low frequency wave with the predetermined frequency.

In one embodiment of the present disclosure, the health care device further includes a block body and at least one negative ion generator. The block body is disposed on the at least one negative ion generator. The negative ion generator is electrically connected to the power supply. A lateral surface of the block body is connected to a metal back plate of the box body, and the block body faces toward a tip of the conical table.

In one embodiment of the present disclosure, the conical table is a wooden cone.

In one embodiment of the present disclosure, the rotating mechanism includes a motor and a cam. The cam is disposed at one side of the motor. The cam is connected to the linkage mechanism. The linkage mechanism includes a connecting rod. The knocker includes a cylinder and a cone. One end of the cylinder is connected to the connecting rod, the other end of the cylinder is connected to the cone, the cylinder is a wooden cylinder, the cone is a wooden cone, and the cylinder and the cone are integrally formed.

In one embodiment of the present disclosure, the knocker further includes a vibrating element, the vibrating element is disposed on a surface of the cylinder or the cone, the vibrating element is electrically connected to the power supply, and the vibrating element vibrates in a thickness direction to generate the bass wave with the predetermined frequency.

In one embodiment of the present disclosure, the block body is a wooden block.

In one embodiment of the present disclosure, at least one surface of the at least one negative ion generator is exposed from the box body.

In one embodiment of the present disclosure, the predetermined frequency is 1.45 Hz.

In one embodiment of the present disclosure, a use of the health care device is configured to transmit the low frequency wave to a spine or a navel.

In the embodiments of the present disclosure, the health care device uses the block body to vibrate and resonate at the low frequency wave transmitted from the tip of the conical table, thereby improving the resonance effect of the box body. In addition, the negative ion generator exposed from the box body generates the negative ion and has the effects of blood purification, cell restoration, and improvement of resistance and self-healing ability of the human body.

The above description of the present disclosure and the following description of the embodiments demonstrate the principles of the present disclosure and provide a further explanation of the claim scope of the present disclosure.

### BRIEF DESCRIPTION OF THE DRAWINGS

FIG. 1 illustrates a stereoscopic view of a health care device according to the first embodiment of the present disclosure.

FIG. 2 illustrates a stereoscopic view of a health care device according to the second embodiment of the present disclosure.

FIG. 3 illustrates a schematic view that the health care device in FIG. 2 is performed according to one embodiment of the present disclosure.

FIG. 4 illustrates a schematic view that the health care device in FIG. 2 is disposed on a frame to be performed according to one embodiment of the present disclosure.

### DETAILED DESCRIPTION OF THE PREFERRED EMBODIMENTS

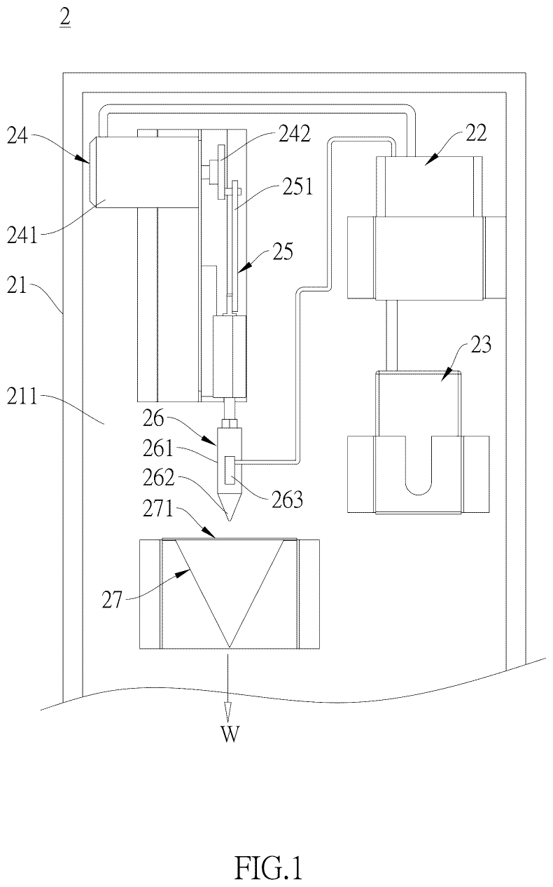

Please refer to FIG. 1, FIG. 1 illustrates a stereoscopic view of a health care device according to the first embodiment of the present disclosure. A health care device 2 in the first embodiment of the present disclosure includes a box body 21, a power supply 22, a speed controller 23, a rotating mechanism 24, a linkage mechanism 25, a knocker 26 and a conical table 27. The box body 21, the power supply 22, the speed controller 23, the rotating mechanism 24, the linkage mechanism 25, the knocker 26 and the conical table 27 are disposed in the box body 21.

In this embodiment, the power supply 22 is electrically connected to the speed controller 23 to output electricity to the speed controller 23. The speed controller 23 is electrically connected to the rotating mechanism 24 and controls the rotating mechanism 24 to rotate at a predetermined speed. The rotating mechanism 24 is connected to the knocker 26 by the linkage mechanism 25. The knocker 26 generates a low frequency wave W with a predetermined frequency, thereby driving the knocker 26 to knock a flat surface 271 of the conical table 27. The conical table 27 is allowed to transmit the low frequency wave W with the predetermined frequency. The predetermined speed causes the knocker 26 to knock the conical table 27 at a knocking frequency. The knocking frequency is in a range from 24 times/per minute to 64 times/per minute.

In this embodiment, the power supply 22 may be a lithium iron phosphate battery. The speed controller 23 may be a device that controls the output speed of a motor 241 by adjusting voltage or current. The box body 21 may include at least one metal back plate 211.

In this embodiment, the rotating mechanism 24 includes the motor 241 and a cam 242. The cam 242 is disposed at one side of the motor 241 and is connected to the linkage mechanism 25. For example, the motor 241 in this embodiment may be a reciprocating motor mechanism.

In this embodiment, the reciprocating motor mechanism rotates reciprocally in the opposite directions, thereby driving the linkage mechanism 25 to reciprocate up and down. The knocker 26 reciprocally knocks the flat surface 271 of the conical table 27.

In this embodiment, the linkage mechanism 25 includes a connecting rod 251. The knocker 26 includes a cylinder 261 and a cone 262. One end of the cylinder 261 is connected to the connecting rod 251, and the other end of the cylinder 261 is connected to the cone 262. The knocker 26 also includes a vibrating element 263. The vibrating element 263 is disposed on the surface of the cylinder 261 or the cone 262. The vibrating element 263 is electrically connected to the power supply 22. The vibrating element 263 is a disc-shaped piezoelectric ceramic transducer. The piezoelectric ceramic transducer is made of a material that includes piezoelectric material polarized in a thickness direction. The power supply 22 provides electricity to the vibrating element 263 to cause the vibrating element 263 to vibrate in the thickness direction. And a bass wave with the predetermined frequency is generated. The predetermined frequency is not greater than 1.81 Hz, or is not less than 1.18 Hz, or is in a range from 1.18 Hz to 1.81 Hz. Optimally, the predetermined frequency is 1.45 Hz.

In other words, the low frequency wave W is the bass wave in this embodiment. The predetermined frequency of the low frequency wave W transmitted when the knocker 26 knocks the conical table 27 is in a range from 1.18 Hz to 1.81 Hz. Preferably, the predetermined frequency of the low frequency wave W transmitted when the knocker 26 knocks the conical table 27 is 1.45 Hz.

In this embodiment, the cone 262 is a wooden cone. The cylinder 261 is a wooden cylinder, and the cylinder 261 and the cone 262 are integrally formed. In addition, the conical table 27 is also a wooden cone.

It is understood that since wood is an elastic body, wood may be vibrated and resonated with the low frequency wave. When the wooden conical table 27 is knocked by the knocker 26, the low frequency wave W will be transmitted. The conical table 27 will resonate with its own resonance or natural frequency. The resonance may emit the bass wave into the air, which may generate a series of low frequency sound waves. Therefore, the box body 21 is a resonance cavity structure, which can cause the low frequency wave W to resonate, thereby amplifying the intensity of the low frequency wave W. Preferably, the box body 21 is a wooden box.

In this embodiment, the low frequency wave W is generated by a series of actions of the power supply 22, the speed controller 23, the rotating mechanism 24, the linkage mechanism 25, the knocker 26 and the conical table 27 disposed in the box body 21. In addition, the resonance effect generated by the resonance cavity structure of the box body 21 can amplify the intensity of the low frequency wave W, thereby enhancing the beneficial effect of the low frequency wave W on the human body.

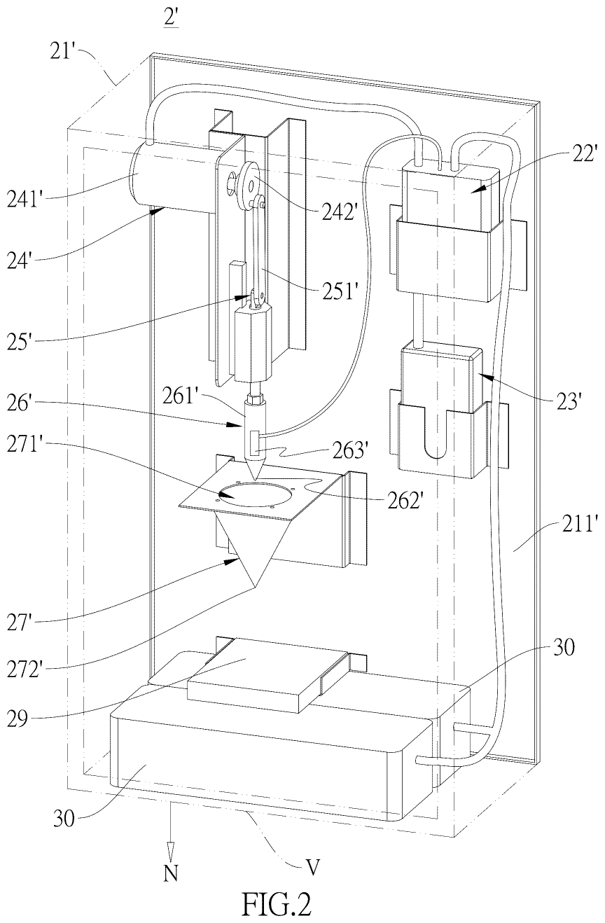

Please refer to FIG. 2, FIG. 2 illustrates a stereoscopic view of a health care device 2′ according to the second embodiment of the present disclosure. The health care device 2′ in the second embodiment of the present disclosure includes a box body 21′, a power supply 22′, a speed controller 23′, a rotating mechanism 24′, a linkage mechanism 25′, a knocker 26′, a conical table 27′, a block body 29 and two negative ion generators 30 disposed in the box body 21′.

In this embodiment, the power supply 22′ is electrically connected to the speed controller 23′ to output electricity to the speed controller 23′. The speed controller 23′ is electrically connected to the rotating mechanism 24′ and controls the rotating mechanism 24′ to rotate at a predetermined speed. The rotating mechanism 24′ is connected to the knocker 26′ through the linkage mechanism 25′ to drive the knocker 26′ to knock a flat surface 271′ of the conical table 27′. Because the low frequency wave W with the predetermined frequency is generated by the knocker 26′, the conical table 27′ may transmit the low frequency wave W with the predetermined frequency.

In this embodiment, the health care device 2′ further includes the block body 29 and the two negative ion generators 30. The negative ion generators 30 are electrically connected to the power supply 22′. The block body 29 is disposed on the two negative ion generators 30. A lateral surface of the block body 29 is connected to a metal back plate 211′, and the block body 29 faces toward a tip 272′ of the conical table 27′.

In this embodiment, the number of the negative ion generators is two, but it is not limited in this regard. The number of negative ion generators may be one, three, or more than three.

In this embodiment, the power supply 22′ may be a lithium iron phosphate battery. The speed controller 23′ may be a device that controls the output speed of a motor 241′ by adjusting voltage or current. The box body 21′ may include the metal back plate 211′, and the block body 29 may be a wooden block.

In this embodiment, at least one surface V of each of the two negative ion generators 30 is exposed from the box body 21′.

In this embodiment, the rotating mechanism 24′ includes the motor 241′ and a cam 242′. The cam 242′ is disposed at one side of the motor 241′, and the cam 242′ is connected to the linkage mechanism 25′.

In this embodiment, the motor 241′ may be a reciprocating motor mechanism. The reciprocating motor mechanism rotates reciprocally in the opposite directions, thereby driving the linkage mechanism 25′ to reciprocate up and down. The knocker 26′ reciprocally knocks the flat surface 271′ of the conical table 27′.

In this embodiment, the linkage mechanism 25′ includes a connecting rod 251′. The knocker 26′ includes a cylinder 261′ and a cone 262′. One end of the cylinder 261′ is connected to the connecting rod 251′, and the other end of the cylinder 261′ is connected to the cone 262′. The knocker 26′ includes a vibrating element 263′ disposed on the surface of the cylinder 261′ or the cone 262′. The vibrating element 263′ is electrically connected to the power supply 22′. The vibrating element 263′ is a disc-shaped piezoelectric ceramic transducer. The piezoelectric ceramic transducer is made of a material that includes piezoelectric material polarized in the thickness direction. The power supply 22′ provides electricity to the vibrating element 263′. The vibrating element 263′ vibrates in the thickness direction to generate the bass wave with the predetermined frequency. The predetermined frequency is not greater than 1.81 Hz, or is not less than 1.18 Hz, or is in a range from 1.18 Hz to 1.81 Hz. Optimally, the predetermined frequency is 1.45 Hz.

In other words, the low frequency wave is the bass wave in this embodiment. The predetermined frequency of the low frequency wave W transmitted when the knocker 26′ knocks the conical table 27′ is in a range from 1.18 Hz to 1.81 Hz. Preferably, the predetermined frequency of the low frequency wave W transmitted when the knocker 26′ knocks the conical table 27′ is 1.45 Hz.

In this embodiment, the cone 262′ is a wooden cone. The cylinder 261′ is a wooden cylinder, and the cylinder 261′ and the cone 262′ are integrally formed. In addition, the conical table 27′ is also a wooden cone. It is understood that since wood is an elastic body, wood may be vibrated and resonated with the low frequency wave. When the wooden conical table 27′ is knocked by the knocker 26′, the low frequency wave W will be transmitted. The conical table 27′ will resonate with its own resonance or natural frequency. The resonance may emit the bass wave into the air, which may generate a series of low frequency sound waves. Therefore, the box body 21′ is a resonance cavity structure, which can cause the low frequency wave W to resonate, thereby amplifying the intensity of the low frequency wave W. Preferably, the box body 21′ is a wooden box.

In this embodiment, the flat surface 271′ of the conical table 27′ is wider and the tip 272′ of the conical table 27′ is narrower, so the conical table 27′ is shaped into a cone. As the low-frequency wave W propagates toward the tip 272′, the energy of the low-frequency wave W becomes more concentrated. Therefore, when the low-frequency wave W is transmitted to the tip 272′ of the conical table 27′, the energy of the low-frequency wave W is concentrated to the maximum. The energy of the low-frequency wave W can be concentrated at the vertex (similar to the principle of “tip discharge” in electricity) and then be transmitted toward the wooden block body 29. The low frequency wave W transmitted from the tip 272′ of the conical table 27′ may generate vibration and resonance on the wooden block body 29. The lateral surface of the wooden block body 29 is connected to the metal back plate 211′, thereby providing better resonance of the box body 21′. Therefore, the knocking frequency may form the low frequency wave W which is intermittent to generate vibration and resonance on the wooden block body 29. The intermittent wave can prevent users from being harmed due to continuous vibration and resonance.

In addition, the negative ions N generated by the negative ion generator 30 exposed from the box body 21′ have the effects of blood purification, cell restoration, enhancement of resistance and self-healing ability, and adjustment of autonomic nerves in the human body.

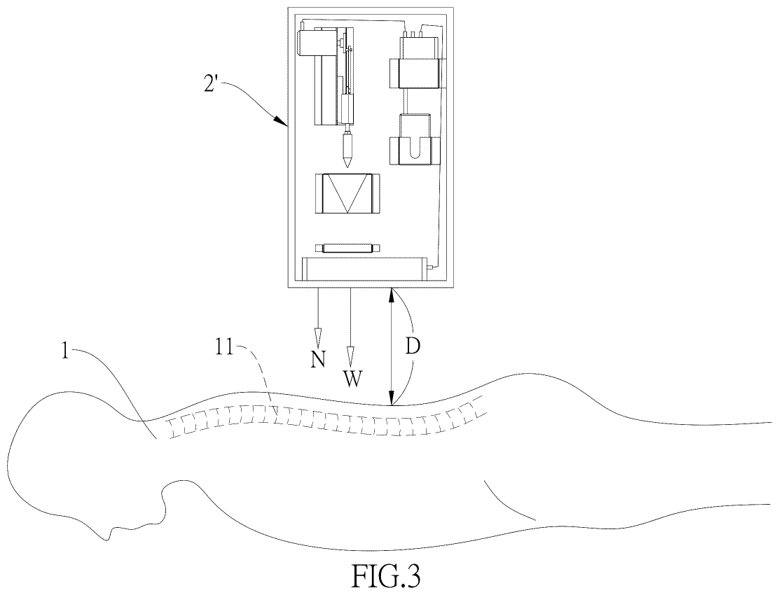

Please refer to FIG. 3. FIG. 3 illustrates a schematic view that the health care device in FIG. 2 is performed according to one embodiment of the present disclosure. The health care device 2′ moves above the spine 11 on the back of the user 1, and the health care device 2′ is located at a predetermined position U with a predetermined distance D from the spine 11. One end of the negative ion generator 30 of the health care device 2′ faces toward the spine 11 on the back of the user 1. Through the operation of the health care device 2, the health care device 2′ transmits the low frequency wave W and negative ions N at the predetermined frequency to the spine 11. The predetermined distance D may be in a range from 0 cm to 8 cm, and the predetermined distance D may be less than or equal to 5 cm, it is not limited in this regard. The predetermined frequency is not greater than 1.81 Hz, or is not less than 1.18 Hz, or is in a range from 1.18 to 1.81 Hz. Optimally, the predetermined frequency is 1.45 Hz.

In this embodiment, the health care device 2′ is configured to perform a health care program to transmit the low frequency wave W and negative ions N to the spines of Person P, Person Q and Person Q′. The predetermined distance D is 0 cm. That is, the health care device 2′ is directly in contact with the spine. The predetermined frequency is 1.45 Hz. The low frequency wave is a bass wave. The negative ion N is 35,000 negative ions per milliliter. The knocking frequency is 24 times/per minute, and this program is executed in 2 hours. Person P is 75 years old and female. Person Q is 72 years old and female. Person Q′ is 76 years old and male. Before performing the health care program, blood samples are taken from Person P, Person Q, and Person Q′. The blood samples are subjected to detect the number of endothelial progenitor cells (EPC) per 2 ml in every blood sample. Within 48 hours after performing the health care program for 80 hours, blood samples are also taken from Person P, Person Q and Person Q′ to detect the number of EPC per 2 ml in every blood sample. The result is shown in Table 1.

TABLE 1

| | number of endothelial progenitor cell | | | |
|---|---|---|---|---|
| inspection | before | after | | |
| item | performing | performing | difference | ratio |
| Person P | 3 | 5 | 2 added | increase 66 |
| Person Q | 3 | 10 | 7 added | increase 233% |
| Person Q′ | 3 | 6 | 3 added | increase 100% |

It can be seen from Table 1 that the number of endothelial progenitor cells of Person P before performing the health care program was 3 cells per 2 ml of blood sample (which is equivalent to a vascular age of 70 years), and after 80 hours of performing the health care program the number of EPC is increased to 5 cells per 2 ml of blood sample (which is equivalent to a vascular age of 60 years). That is, the number of endothelial progenitor cells is increased by 66%. The number of endothelial progenitor cells of Person Q before performing the health care program was 3 cells per 2 ml of blood sample (which is equivalent to a vascular age of 70 years), and after 80 hours of performing the health care program the number of EPC is increased to 10 cells per 2 ml of blood sample (which is equivalent to a vascular age of 50 years). That is, the number of endothelial progenitor cells is increased by 233%. The number of endothelial progenitor cells of Person Q′ before performing the health care program was 3 cells per 2 ml of blood sample (which is equivalent to a vascular age of 70 years), and after 80 hours of performing the health care program the number of EPC is increased to 6 cells per 2 ml of blood sample (which is equivalent to a vascular age of 60 years). That is, the number of endothelial progenitor cells is increased by 100%. Therefore, the health care device may increase the number of endothelial progenitor cells. The health care device may significantly increase the number of endothelial progenitor cells after a two-hour health care program performed within 80 hours.

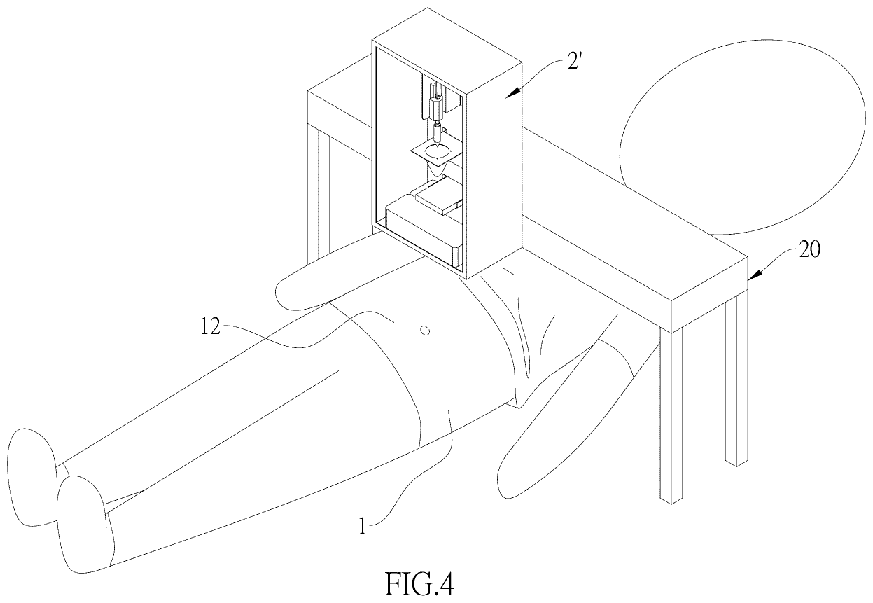

In this embodiment, the health care device 2′ is configured to perform a health care program to transmit the low frequency wave W and negative ions N to the navel 12 (see FIG. 4) of Person R, Person S and Person T. The predetermined distance D is 0 cm. That is, the health care device 2′ is directly in contact with the navel 12. The predetermined frequency is 1.45 Hz. The low frequency wave is a bass wave. The negative ion N is 35,000 negative ions per milliliter. The knocking frequency is 24 times/per minute, and this program is executed in 2 hours. Person R is 76 years old and male. Person S is 72 years old and female. Person T is 44 years old and male. Before performing the health care program, blood samples are taken from Person R, Person S, and Person T. The blood samples are subjected to detect the number of circulating endothelial cells (CEC) per 2 ml in every blood sample. Within 48 hours after performing the health care program for 80 hours, blood samples are also taken from Person R, Person S and Person T to detect the number of CEC per 2 ml in every blood sample. The result is shown in Table 2.

TABLE 2

| | number of circulating endothelial cell | | | |
|---|---|---|---|---|
| inspection | before | after | | |
| item | performing | performing | difference | ratio |
| Person R | 24 | 7 | 17 decreased | decrease 71% |
| Person S | 41 | 26 | 15 decreased | decrease 37% |
| Person T | 34 | 4 | 30 decreased | decrease 88% |

It can be seen from Table 2 that the number of circulating endothelial cells (CEC) of Person R before performing the health care program was 24 cells per 4 ml of blood sample, and after 60 hours of performing the health care program the number of CEC is decreased to 7 cells per 4 ml of blood. That is, the number of CEC is decreased by 71%. The number of CEC of Person S before performing the health care program was 41 cells per 4 ml of blood, and after 60 hours of performing the health care program the number of CEC is decreased to 26 cells per 4 ml of blood sample. That is, the number of CEC is decreased by 37%. The number of CEC of Person T before performing the health care program was 34 cells per 4 ml of blood sample, and after 60 hours of performing the health care program the number of CEC is decreased to 4 cells per 4 ml of blood sample. That is, the number of CEC is decreased by 88%.

As shown in Table 1 and Table 2, the health care device in the present disclosure may increase the number of endothelial progenitor cells and may decrease the number of circulating endothelial cells while performing on human body. The above effects may be achieved after a two-hour program is performed within 80 hours or 60 hours. Because the endothelial progenitor cells (EPC) are the progenitor cells of the vascular endothelial cells and play an important role in angiogenesis and the repair of the vascular endothelial. Therefore, increasing the number of EPC may prevent or mitigate the occurrence of the cardiovascular diseases. In contrast, the circulating endothelial cells (CEC) may be the indicator while the vascular is damaged, which may lead platelets and white blood cells to easily adhere to the wall of the blood vessels. Inflammation and fat accumulation may be generated, and the blood vessels may gradually harden. The inner diameter of blood vessels may gradually decrease. Eventually, thrombosis, arteriosclerosis and other diseases may happen. Therefore, reducing the number of CEC may reduce the risk of cardiovascular-related adverse diseases.

Please refer to FIG. 4, FIG. 4 illustrates a schematic view that the health care device 2′ in FIG. 2 is disposed on a frame 20 to be performed according to one embodiment of the present disclosure. In another embodiment, the health care device 2′ is disposed on a frame 20. The user 1 can lie flat on the platform (not shown). The frame 20 may be disposed above the platform. The health care device 2′ may be located above the spine 11 or navel 12 of the user 1. In this way, the user 1 can get proper rest during the health care program.

It is particularly noted that the above bass wave is only an example of a low frequency wave, and the present disclosure is not limited in this regard. The low frequency wave can be an electromagnetic wave such as an electric wave or a light wave.

The above is only a detailed description of the present disclosure through preferred embodiments. Simple modifications and changes made to the embodiments shall fall within the spirit and scope of the present disclosure.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. A health care device, comprising:
a box body;
a power supply;
a speed controller;
a rotating mechanism;
a linkage mechanism;
a knocker; and
a conical table, wherein the power supply, the speed controller, the rotating mechanism, the linkage mechanism, the knocker and the conical table are disposed in the box body;
wherein the power supply is electrically connected to the speed controller to output electricity to the speed controller, the speed controller is electrically connected to the rotating mechanism and controls the rotating mechanism to rotate at a predetermined speed, the predetermined speed causes the knocker to knock the conical table at a knocking frequency, the knocking frequency is in a range from 24 times/per minute to 64 times/per minute, the knocker generates a low frequency wave with a predetermined frequency, the predetermined frequency is in a range from 1.18 Hz to 1.81 Hz, the low frequency wave is a bass wave, the rotating mechanism is connected to the knocker by the linkage mechanism to drive the knocker to knock a flat surface of the conical table, such that the conical table transmits the low frequency wave with the predetermined frequency.

2. The health care device of claim 1, further comprising:
a block body; and
at least one negative ion generator, wherein the block body is disposed on the at least one negative ion generator, the negative ion generator is electrically connected to the power supply, a lateral surface of the block body is connected to a metal back plate of the box body, and the block body faces toward a tip of the conical table.

3. The health care device of claim 2, wherein the conical table is a wooden cone.

4. The health care device of claim 3, wherein the rotating mechanism comprises a motor and a cam, the cam is disposed at one side of the motor, the cam is connected to the linkage mechanism, the linkage mechanism comprises a connecting rod, the knocker comprises a cylinder and a cone, one end of the cylinder is connected to the connecting rod, the other end of the cylinder is connected to the cone, the cylinder is a wooden cylinder, the cone is a wooden cone, and the cylinder and the cone are integrally formed.

5. The health care device of claim 4, wherein the knocker further comprises a vibrating element, the vibrating element is disposed on a surface of the cylinder or the cone, the vibrating element is electrically connected to the power supply, and the vibrating element vibrates in a thickness direction to generate the bass wave with the predetermined frequency.

6. The health care device of claim 5, wherein the block body is a wooden block.

7. The health care device of claim 6, wherein at least one surface of the at least one negative ion generator is exposed from the box body.

8. The health care device of claim 7, wherein the predetermined frequency is 1.45 Hz.

9. A use of the health care device of claim 1 for transmitting the low frequency wave to a spine or a navel.
